(19) United States
(12) Patent Application Publication
(10) Pub. No.: US 2026/0115772 A1
(43) Pub. Date: Apr. 30, 2026

(54) FOOD WASTE DISPOSER WITH AIR EXHAUST DUCT CAPABLE OF BEING DISASSEMBLED, ASSEMBLED AND CLEANED CONVENIENTLY

(71) Applicant: Guangdong Shihao Intelligent Technology Co. , Ltd., Foshan (CN)

(72) Inventors: Chuanliang Zheng, Foshan (CN); Jianwei Liu, Foshan (CN); Qing Xiao, Foshan (CN); Wennian Li, Foshan (CN); Tao Zheng, Foshan (CN)

(21) Appl. No.: 18/949,905

(22) Filed: Nov. 15, 2024

(30) Foreign Application Priority Data
Oct. 30, 2024 (CN) 202422650738.4

(51) Int. Cl. B09B 3/32; B09B 3/40; B09B 101/70

(52) U.S. Cl. CPC B09B 3/32; B09B 3/40; B09B 2101/70

(57) ABSTRACT

A food waste disposer with an air exhaust duct capable of being disassembled, assembled and cleaned conveniently comprises a main casing, a drying and crushing device, an exhaust deodorizing device, a coil core assembly, and a magnetic rotor. The main casing comprises a device chamber, a deodorizing chamber with a rear opening, and a detachable back cover. The exhaust deodorizing device comprises a water receiving bottom box, a ventilation device, and a deodorizing box. The ventilation device comprises a longitudinal air duct and a centrifugal wind wheel. The longitudinal air duct comprises an air suction duct, a volute, and an air exhaust duct. The air suction duct is communicated with an air guide channel. The centrifugal wind wheel and the magnetic rotor are provided on the ventilation device, and the magnetic rotor rotates with the centrifugal wind wheel. During cleaning, there is no need to worry about electrical conductivity.

### CROSS-REFERENCE TO RELATED APPLICATIONS

This is a U.S. patent application which claims the priority and benefit of Chinese Patent Application Number 202422650738.4, filed on Oct. 30, 2024, the disclosure of which is incorporated herein by reference in its entirety.

### TECHNICAL FIELD

The present invention relates to a food waste disposer.

### BACKGROUND

According to current existing technologies, a Chinese Patent 202120598363.5 provides a food waste disposer, comprising: a machine body and a cover body covered on the machine body, wherein a disposal barrel is provided in the machine body, and an air inlet and an air outlet are also formed on an outer wall of the machine body; a hot air assembly, wherein the hot air assembly comprises an air duct provided in the machine body, the air duct is communicated with the inside of the disposal barrel, an air inlet end of the air duct is communicated with the air inlet, a heating body and an air exhaust fan are provided in the air duct, and an air outlet end of the air duct is communicated with the air outlet; and a purifying assembly, wherein the purifying assembly is arranged in the air duct and used for performing purifying treatment on gas discharged from the disposal barrel. The food waste disposer has the problems that a power supply wire is required, the power supply wire is easily exposed to water during cleaning, and the air duct is inconvenient to disassemble and assemble.

### SUMMARY

The purpose of the present invention is to provide a food waste disposer with an air exhaust duct capable of being disassembled, assembled and cleaned conveniently, which has a ventilation device and is convenient to clean without troubles.

The present invention is implemented as follows: a food waste disposer with an air exhaust duct capable of being disassembled, assembled and cleaned conveniently, characterized by comprising a main casing, a drying and crushing device, an exhaust deodorizing device, a coil core assembly, and a magnetic rotor;
the main casing comprises a device chamber, a deodorizing chamber with a rear opening, and a detachable back cover, and the device chamber is communicated with the deodorizing chamber through an air guide channel; a partition plate between the device chamber and the deodorizing chamber is provided with a stator chamber with one opening end and a shaft sleeve provided in the stator chamber, the opening end of the stator chamber is located in the device chamber, and an opening end of the shaft sleeve is located in the deodorizing chamber; and the back cover is in detachable fit with the deodorizing chamber;
the exhaust deodorizing device comprises a water receiving bottom box, a ventilation device, and a deodorizing box, and the ventilation device is in detachable fit with the deodorizing box and the water receiving bottom box;
the ventilation device comprises a longitudinal air duct and a centrifugal wind wheel, the longitudinal air duct comprises an air suction duct, a volute, and an air exhaust duct, the volute comprises a cylindrical seat with a side opening, a shaft seat, an inlet hole, and an air outlet, an air inlet hole is communicated with the air suction duct, and the air outlet is communicated with the air exhaust duct;
the coil core assembly is provided in the stator chamber and surrounds the shaft seat, the magnetic rotor is provided around the cylindrical seat, a rotating shaft of the centrifugal wind wheel is in rotating fit with the shaft seat, and the magnetic rotor rotates with the centrifugal wind wheel; the exhaust deodorizing device is in detachable fit with the deodorizing chamber, the stator chamber is in detachable fit with the cylindrical seat, and the shaft seat is in detachable fit with the shaft sleeve; and the air suction duct is communicated with the air guide channel.

The food waste disposer with an air exhaust duct capable of being disassembled, assembled and cleaned conveniently is characterized in that the rotating shaft of the centrifugal wind wheel is in rotating fit with the shaft seat through a bearing.

The food waste disposer with an air exhaust duct capable of being disassembled, assembled and cleaned conveniently is characterized in that the centrifugal wind wheel comprises the rotating shaft, a wheel hub, an annular frame plate, and blades;
the wheel hub is provided with one opening end and comprises an annular seat plate provided at the opening end and a shaft fixing seat;
the rotating shaft is fixed to the shaft fixing seat, and two ends of the blades are fixed to the annular frame plate and the annular seat plate, respectively; and
the magnetic rotor is fixed around the wheel hub, and the cylindrical seat extends into the wheel hub and is in clearance fit or rotating fit with the magnetic rotor.

The food waste disposer with an air exhaust duct capable of being disassembled, assembled and cleaned conveniently is characterized in that a bottom plate of the deodorizing chamber is provided with a guide rail; and
a bottom plate of the water receiving bottom box is provided with a track, and the guide rail and the track are positioned in an engaged mode and are in sliding fit.

The food waste disposer with an air exhaust duct capable of being disassembled, assembled and cleaned conveniently is characterized in that a top plate of an opening end of the deodorizing chamber is provided with a hook hole, and a bottom plate is provided with a pin hole;
the back cover comprises a pin plate provided at the bottom, an elastic hook provided at the top, and an air outlet chamber, and the air outlet chamber is provided with an air outlet hole group;
the deodorizing box is provided with an air outlet head; and
the pin plate is inserted into the pin hole, a hook part of the elastic hook is in fit with the hook hole, and the air outlet head extends into the air outlet chamber and is in detachable fit.

According to the food waste disposer with an air exhaust duct capable of being disassembled, assembled and cleaned conveniently of the present invention, the centrifugal wind wheel and the magnetic rotor are provided on the ventilation device, and the magnetic rotor rotates with the centrifugal wind wheel. During cleaning, there is no need to worry about the problem of electrical conductivity.

### BRIEF DESCRIPTION OF DRAWINGS

FIG. 1 is a section view of the present invention.

FIG. 2 is a planar exploded view of the present invention.

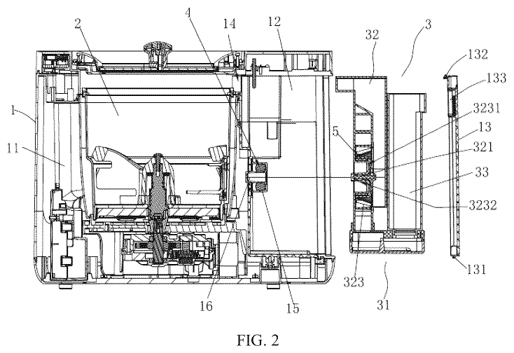

FIG. 3 is a stereoscopic view of the present invention.

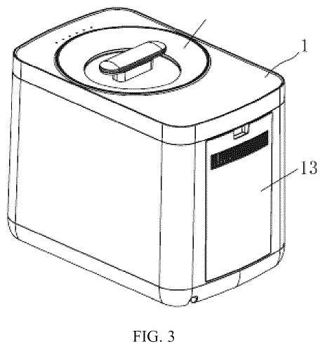

FIG. 4 is a first stereoscopic exploded view of the present invention.

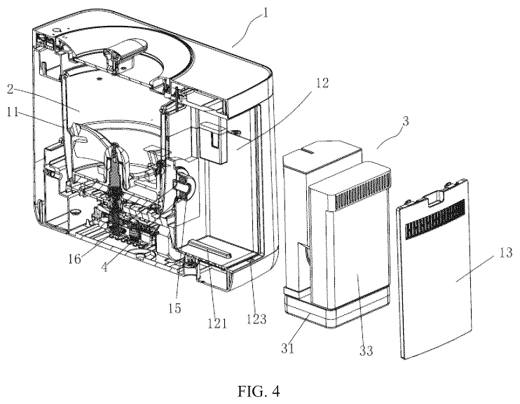

FIG. 5 is a second stereoscopic exploded view of the present invention.

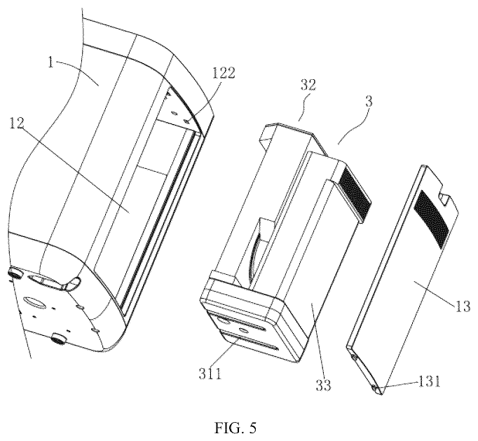

FIG. 6 is a section view of an exhaust deodorizing device of the present invention.

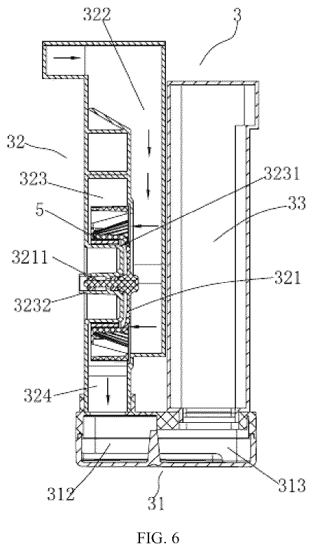

FIG. 7 is a stereoscopic view of the exhaust deodorizing device of the present invention.

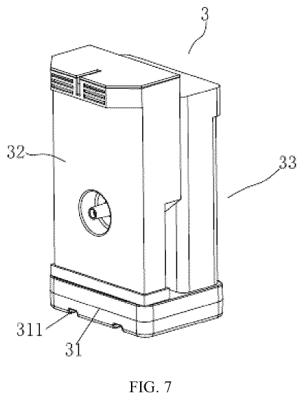

FIG. 8 is a first stereoscopic exploded view of the exhaust deodorizing device of the present invention.

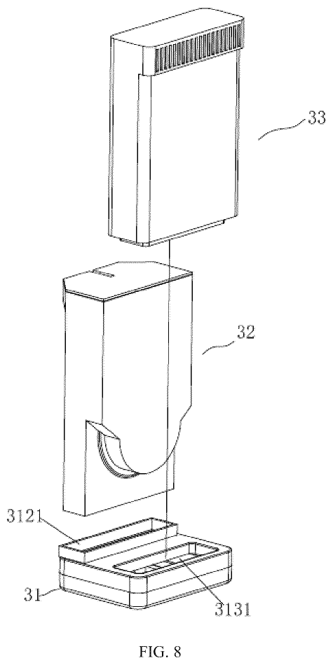

FIG. 9 is a second stereoscopic exploded view of the exhaust deodorizing device of the present invention.

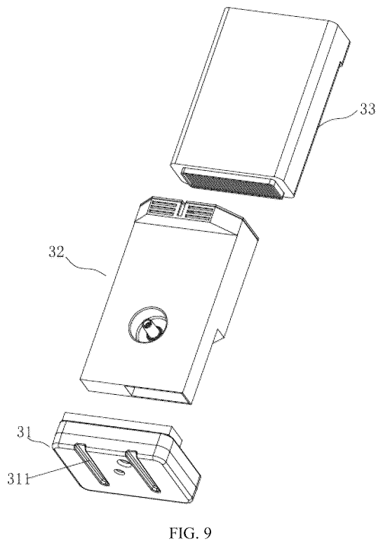

FIG. 10 is a first stereoscopic view of a centrifugal wind wheel of the present invention.

FIG. 11 is a second stereoscopic view of the centrifugal wind wheel of the present invention.

### DESCRIPTION OF EMBODIMENTS

The embodiments of the present invention are described in detail below, and examples of the embodiments are illustrated in the drawings, in which same or similar reference numerals throughout represent same or similar elements or elements with same or similar functions. The embodiments described below with reference to the drawings are exemplary, which are only used to explain the present invention and cannot be understood as limiting the present invention.

In the description of the present invention, it should be noted that, unless otherwise clearly stated and limited, terms, such as “mount”, “connect”, and “connection” should be understood in a broad sense, for example, may be fixed connection, and may also be detachable connection or integral connection; and may be direct connection, and may also be indirect connection through an intermediate medium, or internal communication between two elements. For persons of ordinary skill in the art, specific meanings of the above terms in the present invention may be understood according to specific circumstances.

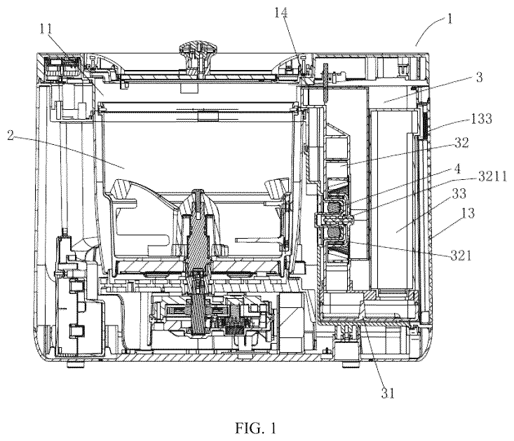

As shown in FIG. 1, a food waste disposer with an air exhaust duct capable of being disassembled, assembled and cleaned conveniently comprises a main casing 1, a drying and crushing device 2, an exhaust deodorizing device 3, a coil core assembly 4, and a magnetic rotor 5;
the main casing 1 comprises a device chamber 11, a deodorizing chamber 12 with a rear opening, and a detachable back cover 13, and the device chamber 11 is communicated with the deodorizing chamber 12 through an air guide channel 14; a partition plate between the device chamber 11 and the deodorizing chamber 12 is provided with a stator chamber 15 with one opening end and a shaft sleeve 16 provided in the stator chamber 15, the opening end of the stator chamber 15 is located in the device chamber 11, and an opening end of the shaft sleeve 16 is located in the deodorizing chamber 12; the back cover 13 is in detachable fit with the deodorizing chamber 12; the device chamber 11 is provided with a top opening and a chamber cover; the drying and crushing device 2 is provided in the device chamber 11 and is opposite to the top opening of the device chamber 11; the chamber cover is hinged with the main casing and is in fit with the top opening;
the exhaust deodorizing device 3 comprises a water receiving bottom box 31, a ventilation device 32, and a deodorizing box 33, and activated carbon is provided in the deodorizing box; the ventilation device 32 is in detachable fit with the deodorizing box 33 and the water receiving bottom box 31;
the ventilation device 32 comprises a longitudinal air duct and a centrifugal wind wheel 321, the longitudinal air duct comprises an air suction duct 322, a volute 323, and an air exhaust duct 324, the volute 323 comprises a cylindrical seat 3231 with a side opening, a shaft seat 3232, an inlet hole, and an air outlet, an air inlet hole is communicated with a lower part of the air suction duct 322, and the air outlet is communicated with the air exhaust duct 324; the water receiving bottom box 31 comprises a condensate water chamber 312 and
an air outlet chamber 313, the condensate water chamber 312 is communicated with the air outlet chamber 313, a top plate of the condensate water chamber 312 is provided with an air inlet seat 3121 with upper and lower openings, the air inlet seat 3121 is provided with an internal connecting port, and a top plate of the air outlet chamber 313 is provided with an insertion hole 3131;
a lower end of the deodorizing box 33 is provided with an external connecting port;
the air exhaust duct 324 is in detachable fit with the internal connecting port, and the external connecting port is in detachable fit with the insertion hole 3131;
the coil core assembly 4 is provided in the stator chamber 15 and surrounds the shaft seat 3232, the magnetic rotor 5 is provided around the cylindrical seat 3231, a rotating shaft 3211 of the centrifugal wind wheel 321 is in rotating fit with the shaft seat 3232, and the magnetic rotor 5 rotates with the centrifugal wind wheel 321; the exhaust deodorizing device 3 is in detachable fit with the deodorizing chamber 12, the stator chamber 15 is in detachable fit with the cylindrical seat 3231, and the shaft seat 3232 is in detachable fit with the shaft sleeve 16; and the air suction duct 322 is communicated with the air guide channel 14.

By adopting such structure, when the ventilation device 32 is only provided with the magnetic rotor of a motor without a power supply connection terminal, functions and power supply wiring properties of the motor are not affected after cleaning.

As a further improvement of the present invention: the rotating shaft of the centrifugal wind wheel 321 is in rotating fit with the shaft seat 3232 through a bearing, the bearing is provided in the shaft seat 3232, and the rotating shaft is fixed to an inner ring of the bearing.

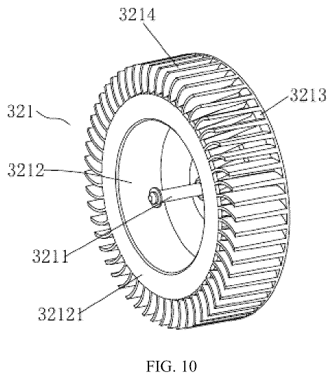

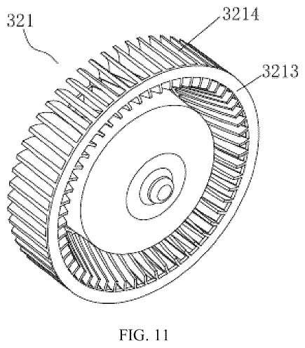

As shown in FIG. 10 and FIG. 11, the centrifugal wind wheel 321 comprises the rotating shaft 3211, a wheel hub 3212, an annular frame plate 3213, and blades 3214;
the wheel hub 3212 is provided with one opening end and comprises an annular seat plate 32121 provided at the opening end and a shaft fixing seat;
the rotating shaft 3211 is fixed to the shaft fixing seat, and two ends of the blades 3214 are fixed to the annular frame plate 3213 and the annular seat plate 32121, respectively; and
the magnetic rotor 5 is fixed around the wheel hub 3212, and the cylindrical seat 3231 extends into the wheel hub 3212 and is in clearance fit or rotating fit with the magnetic rotor 5.

As a further improvement of the present invention: a bottom plate of the deodorizing chamber 12 is provided with a guide rail 121; and
a bottom plate of the water receiving bottom box 31 is provided with a track 311, and the guide rail 121 and the track 311 are positioned in an engaged mode and are in sliding fit.

As a further improvement of the present invention: a top plate of an opening end of the deodorizing chamber 12 is provided with a hook hole 122, and a bottom plate is provided with a pin hole 123;
the back cover 13 comprises a pin plate 131 provided at the bottom, an elastic hook 132 provided at the top, and an air outlet chamber 133, and the air outlet chamber 133 is provided with an air outlet hole group;
the air suction duct 322 is provided with an air outlet head; and
the pin plate 131 is inserted into the pin hole 123, a hook part of the elastic hook 132 is in fit with the hook hole 122, and the air outlet head extends into the air outlet chamber and is in detachable fit.

Although the embodiments of the present invention are shown and described, it may be understood by those of ordinary skill in the art that various changes, modifications, substitutions and alternations of the embodiments may be made without departing from the principles and spirit of the present invention, and the scope of the present invention is defined by the claims and equivalents thereof.

What is claimed is:

1. A food waste disposer with an air exhaust duct capable of being disassembled, assembled and cleaned conveniently, comprising a main casing, a drying and crushing device, an exhaust deodorizing device, a coil core assembly, and a magnetic rotor;
the main casing comprises a device chamber, a deodorizing chamber with a rear opening, and a detachable back cover, and the device chamber is communicated with the deodorizing chamber through an air guide channel; a partition plate between the device chamber and the deodorizing chamber is provided with a stator chamber with one opening end and a shaft sleeve provided in the stator chamber, the opening end of the stator chamber is located in the device chamber, and an opening end of the shaft sleeve is located in the deodorizing chamber; and the back cover is in detachable fit with the deodorizing chamber;
the exhaust deodorizing device comprises a water receiving bottom box, a ventilation device, and a deodorizing box, and the ventilation device is in detachable fit with the deodorizing box and the water receiving bottom box;
the ventilation device comprises a longitudinal air duct and a centrifugal wind wheel, the longitudinal air duct comprises an air suction duct, a volute, and an air exhaust duct, the volute comprises a cylindrical seat with a side opening, a shaft seat, an inlet hole, and an air outlet, an air inlet hole is communicated with the air suction duct, and the air outlet is communicated with the air exhaust duct;
the coil core assembly is provided in the stator chamber and surrounds the shaft seat, the magnetic rotor is provided around the cylindrical seat, a rotating shaft of the centrifugal wind wheel is in rotating fit with the shaft seat, and the magnetic rotor rotates with the centrifugal wind wheel; the exhaust deodorizing device is in detachable fit with the deodorizing chamber, the stator chamber is in detachable fit with the cylindrical seat, and the shaft seat is in detachable fit with the shaft sleeve; and the air suction duct is communicated with the air guide channel.

2. The food waste disposer with an air exhaust duct capable of being disassembled, assembled and cleaned conveniently according to claim 1, wherein the rotating shaft of the centrifugal wind wheel is in rotating fit with the shaft seat through a bearing.

3. The food waste disposer with an air exhaust duct capable of being disassembled, assembled and cleaned conveniently according to claim 1, wherein the centrifugal wind wheel comprises the rotating shaft, a wheel hub, an annular frame plate, and blades;
the wheel hub is provided with one opening end and comprises an annular seat plate provided at the opening end and a shaft fixing seat;
the rotating shaft is fixed to the shaft fixing seat, and two ends of the blades are fixed to the annular frame plate and the annular seat plate, respectively; and
the magnetic rotor is fixed around the wheel hub, and the cylindrical seat extends into the wheel hub and is in clearance fit or rotating fit with the magnetic rotor.

4. The food waste disposer with an air exhaust duct capable of being disassembled, assembled and cleaned conveniently according to claim 1, wherein a bottom plate of the deodorizing chamber is provided with a guide rail; and
a bottom plate of the water receiving bottom box is provided with a track, and the guide rail and the track are positioned in an engaged mode and are in sliding fit.

5. The food waste disposer with an air exhaust duct capable of being disassembled, assembled and cleaned conveniently according to claim 1, wherein a top plate of an opening end of the deodorizing chamber is provided with a hook hole, and a bottom plate is provided with a pin hole;
the back cover comprises a pin plate provided at the bottom, an elastic hook provided at the top, and an air outlet chamber, and the air outlet chamber is provided with an air outlet hole group;
the deodorizing box is provided with an air outlet head; and
the pin plate is inserted into the pin hole, a hook part of the elastic hook is in fit with the hook hole, and the air outlet head extends into the air outlet chamber and is in detachable fit.
